(19) United States
(12) Patent Application Publication
(10) Pub. No.: US 2004/0234954 A1
(43) Pub. Date: Nov. 25, 2004

(54) Surfaces that selectively bind to moieties

(76) Inventors: Klaus Nusslein, Amherst, MA (US); Jacques Penelle, Verrieres-le-Buisson (FR); Kanad Das, San Francisco, CA (US)

Correspondence Address: FISH & RICHARDSON PC, 225 FRANKLIN ST, BOSTON, MA, 02110, US

(21) Appl. No.: 10/795,703

(22) Filed: Mar. 3, 2004

Related U.S. Application Data
(60) Provisional application No. 60/451,828, filed on Mar. 3, 2003.

(51) Int. Cl. C12Q 1/70; C12M 1/34; C12Q 1/18

(52) U.S. Cl. 435/5; 424/400; 435/32; 435/287.1

(57) ABSTRACT

A thin film made of a stiff polymer and having indentations on its surface, and methods of making and using the thin film are disclosed. The surface includes a plurality of indentations, the shape of each indentation corresponding to an outer surface of an imprint moiety, such as an animal, bacterial, or plant cell. The thin film can be mounted on a detector, which can be used to selectively bind to and detect the imprint moiety in a sample, e.g., a biological or environmental sample, at a minimum concentration of as low as 500 to 1000 imprint moieties per milliliter of sample.

### CROSS REFERENCE TO RELATED APPLICATIONS

This application claims the benefit of priority from U.S. Provisional Patent Application No. 60/451,828, filed on Mar. 3, 2003, the contents of which is incorporated herein by reference in its entirety.

### STATEMENT AS TO FEDERALLY SPONSORED RESEARCH

This invention was made with Government support under Grant Nos. 0085495, and DMR-9809365 awarded by National Science Foundation. The Government thus has certain rights in the invention.

### TECHNICAL FIELD

This invention relates to methods of making an imprinted surface, and methods for detecting imprint moieties, such as cells, and virus particles on an imprinted surface.

### BACKGROUND

Techniques for the capture, isolation, detection, analysis, and quantification of imprint moieties, such as animal, bacterial, and plant cells, in environments, for example, air, soil, skin, biological fluids, housing, mass transportation systems, and hospitals are needed. Methodologies based on polymerase chain-reaction (PCR) and immunoassay methodologies have been explored to increase speed and sensitivity for detecting imprint moieties, but require highly experienced personnel and expensive equipment. These methods for detecting imprint moieties are cumbersome, and not amenable to continuous real-time monitoring. A rapid and cost-effective method for detecting and localizing imprint moieties on surfaces is needed.

### SUMMARY

The invention is based, in part, on the discovery that thin films made of stiff polymers having a flexural modulus of at least 150,000 psi and a maximum thickness of 2 microns can be used in sensors, such as a quartz crystal microbalance, to selectively and rapidly detect imprint moieties in biological and environmental samples at concentrations as low as 100 to 1000 cells or particles per milliliter of sample. Imprint moieties include, for example, mammalian, plant, and bacterial cells and spores, as well as other microorganisms such as fungi, algae, and virus particles. The new thin films have numerous indentations (binding sites) on their surface, where each indentation is a reverse impression (e.g., a negative mold) of a portion of an outer surface of an imprint moiety. These indentations allow the thin film to selectively bind to specific imprint moieties at environmentally relevant concentrations.

In addition, the new thin films can be attached to the surface of implantable medical devices, such as artificial joints or organs, to help stimulate the efficient attachment and growth of human body cells, e.g., endothelial cells, to the device.

In general, the invention features thin films that have a maximum thickness of 2 microns and are comprised of a polymer having a flexural modulus of at least 150,000 psi, wherein a surface of the film has a plurality of indentations, each indentation being a reverse impression of (e.g., a negative mold or cavity that corresponds to) a portion of an outer surface of an imprint moiety. When the thin film is mounted on a quartz crystal microbalance (QCM) as described herein, the QCM can detect the imprint moiety in a liquid sample at a minimum concentration of at least as low as 1000 (e.g., as low as 750, 500, 250, or 100) imprint moieties per milliliter of sample.

In some implementations, the imprint moieties are, for example, bacteria, virus particles, animal cells, spores, plant cells, prokaryotic cells, and eukaryotic cells. In some instances, the bacterium can be Escherichia coli, Staphylococcus aureus, or Bacillus megaterium, or other relevant pathogenic microorganisms.

In some embodiments, the maximum thickness of the film is, for example, between about 100 nm and about 1500 nm, e.g., 250, 500, 750, 1000, or 1250 nm. For some applications, the flexural modulus is at least 180,000, 200,000, or 225,000 psi or higher, for example, at least 250,000 psi.

For some applications, the QCM can detect the imprint moiety in a liquid sample at a minimum concentration of at least as low as 500 imprint moieties per milliliter of sample. The indentations can have, for example, a maximum depth of from about 20% to about 40% of a largest dimension of the imprint moiety.

In some instances, the indentations on the surface number from about 40,000 to about 150,000 indentations/cm² or from about 60,000 to about 90,000 indentations/cm².

The polymer can include, for example, a bis-acrylate polymer, a methacrylate, an acrylate polymer, or blends or combinations thereof. In some applications, the polymer is formed by polymerization of a mixture of 1,5-pentanediol bis(α-acetamido acrylate), and benzyl methacrylate or benzyl acrylate. In some instances, the polymer is formed by polymerization of a monomer selected from the group of acrylic acid, methyl acrylate, ethyl acrylate, butyl acrylate, 2-ethylhexyl acrylate, methacrylic acid, methylmethacrylate, isobutyl acrylate, tertiarybutyl acrylate, tertiarybutyl methacrylate, 2-hydroxyethyl acrylate, 2-hydroxypropyl acrylate, butanediol monoacrylate, ethyldiglycol acrylate, lauryl acrylate, dimethylaminoethyl acrylate, dihydrodicyclopentadienyl acrylate, and mixtures thereof.

The polymers can also include nylons, polyesters, and polycarbonates, as long as they have a flexural modulus of at least 150,000 psi.

In another aspect, the invention features an implantable medical device that includes one or more of the new thin films described herein attached to at least a portion of its surface.

In some embodiments, the imprint moiety is a mammalian cell. In some applications, the mammalian cell is from a mammal into which the medical device is to be implanted. The mammalian cell is, for example, an endothelial cell or a cartilage cell.

In another aspect, the invention features a biosensor that includes a microbalance that includes a conducting element, and one of the new thin films described herein, attached to a surface of the conducting element. In some implementations, the microbalance is a quartz crystal microbalance. For some applications, the thickness of the film is from about 100 nm to 1500 nm, or the thickness of the film is less than 500 nm. For some applications, the imprint moiety is, for example, a bacterium, a virus particle, a cell from an animal, a spore, a plant cell, a prokaryotic cell, or a eukaryotic cell.

In another aspect, the invention features a method of detecting a target imprint moiety in a sample. The method includes obtaining a biosensor of as described herein. The thin film includes indentations that are reverse impressions of the target imprint moiety to be detected. A sample is applied to the thin film on the biosensor under conditions that enable the thin film to selectively bind to any target imprint moieties in the sample. The detector can detect the target imprint moiety at a minimum concentration of at least as low as 1000 imprint moieties per milliliter of sample. Any change in mass of the thin film is detected, and an increase in mass of the thin film indicates the presence of the imprint moiety in the sample.

In some embodiments, the sample is, for example, blood, sputum, saliva, urine, or serum. In some applications, the target imprint moiety is, for example, a bacterium, a virus particle, an animal cell, a spore, a plant cell, a prokaryotic cell, or a eukaryotic cell.

In another aspect, the invention features methods of making the new thin films. The methods include polymerizing one or more monomers in the presence of a plurality of imprint moieties to form a polymer having a flex modulus of at least 150,000 psi. A sheet is formed from the polymer having a maximum thickness of 2 microns. The imprint moieties are removed from a surface of the polymer sheet leaving a plurality of indentations on the surface, each indentation comprising a reverse impression of a portion of an outer surface of an imprint moiety. The thin film can be then mounted on a quartz crystal microbalance (QCM), and the QCM can detect the imprint moiety in a liquid sample at a minimum concentration of at least as low as 1000 imprint moieties per milliliter of sample.

In some implementations, all of the imprint moieties are of the same type. For some applications, the imprint moieties are of two or more different types.

The invention provides several advantages. The new thin films can be tailored to a particular application based on the types of imprint moiety, for example, type of cell, cell shape, size, cell wall composition, concentration of cells in the environmental medium, types of materials, e.g., monomers used, thickness of the polymer film, and concentration of indentations or binding sites on the polymer film. The imprints of the imprint moieties in the surface of the thin films provide a binding site for the same type of imprint moiety on the surface. The thin films, when placed on a sensor of a detector, can detect imprint moieties directly in environmental samples rapidly, e.g., in real time, for example, in seconds and up to about 5 minutes, rather than minutes and hours.

Furthermore, the new system can detect imprint moieties at environmentally relevant concentrations (e.g., 100 to 500 imprint moieties/mL, or 500 to 1000 imprint moieties/mL) without the need for sample treatment, e.g., concentration of the sample or purification.

Without being bound by any particular theory, it is believed that enhanced sensitivity, for example, the ability to detect environmentally relevant concentrations (e.g., 100 to 500 imprint moieties/mL, or 500 to 1000 imprint moieties/mL) arises from the fact that the films are thin, e.g., from about 100 nm to about 1500 nm or more, e.g., 250, 500, 750, 1000 nm or more, e.g., 2000 nm. It is believed that the thinness of the film plays two roles in enhancing sensitivity. First, the thinness of the film places the imprint moieties close to a detector surface (e.g., a conducting surface of a microbalance), thus improving modulation of mass differences since a captured imprint moiety is in close proximity to the detector. Second, thinner films have a lower vibration dampening factor, and, as a consequence, thinner films tend to modulate mass differences better.

In addition, it is believed that the selection of the stiff polymer with a flexural modulus greater than about 150,000 psi, e.g., acrylate and methacrylate polymers, nylons, polyesters, or polycarbonates, help to improve modulation of mass differences, and as a result, help to improve sensitivity.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1a and 1b are schematics illustrating the preparation of a thin polymer film for molecular imprinting of an imprint moiety, removal of the imprint moiety, and later detection of the same imprint moiety type.

FIGS. 2a, 2b, and 2c are graphs illustrating the measured quartz crystal microbalance response to a 500 cells/mL saline solution.

FIGS. 3a, 3b, and 3c are optical micrographs of the cells of three unrelated bacterial species, which in the case of E. coli (A) occur as discrete objects, in the case of B. megaterium (B) occur as dimers and trimers, and in the case of S. aureus (C) occur as aggregates, in saline.

FIGS. 4a and 4b are graphs illustrating the observed frequency shifts of surfaces imprinted with B. megaterium (4A) and E. coli (4B) compared to imprinted and unimprinted cell types.

FIGS. 5a, 5b, 5c, and 5d are scanning electron micrographs (SEMs) of a thin polymer film surface during sensor fabrication, and testing.

FIG. 6 is a schematic illustrating an experimental setup for detection using a quartz crystal microbalance chip.

### DETAILED DESCRIPTION

Described herein, in several aspects, is molecular imprinting on a surface, e.g., a thin polymer film. We have discovered that polymers can be used to prepare an imprinted surface, e.g., a thin polymer film, with a high number of indentations or binding sites for specific imprint moieties. The imprinted surface, e.g., thin polymer films are, for example, prepared by ultraviolet or thermal polymerization of a monomer mixture in the presence of specific imprint moieties. For example, the monomers can be 1,5-pentanediol bis(α-acetamido acrylate) and benzyl methacrylate. The imprint moieties are removed to leave binding sites on the imprinted thin polymer films. The imprinted thin polymer films can be applied to or created on sensors of detection devices to selectively detect the specific imprint moieties, because the films selectively bind with the specific imprint moieties.

The new methods can be used to detect or localize the presence of imprint moieties in a variety of pharmaceutical, medical diagnostics, and industrial uses. For example, biosensors fabricated from the films can be used to detect pathogenic imprint moieties, e.g., for medical diagnostics, or to provide a first line of defense against bio-terrorists attacks. For example, the films can be used to detect imprint moieties in environmental samples, food and pharmaceutical processes, and to make surfaces attractive to imprint moieties, e.g., during fabrication of prosthetics.

Thin Polymer Films

The invention includes new imprinted polymer films and new methods of making the imprinted polymer films are described. The films can include monomers, for example, acrylates (e.g., bis acrylates), methacrylates (e.g., aryl methacrylate), vinyl ethers, vinyl sulfides, allyls, bicyclic enes, acetylenes, epoxides, styrenes, and acrylamides. Specific acrylates and methacrylates (e.g., available from BASF) include, for example, acrylic acid, methyl acrylate, ethyl acrylate, butyl acrylate, 2-ethylhexyl acrylate, methacrylic acid, methylmethacrylate, isobutyl acrylate, tertiarybutyl acrylate, tertiarybutyl methacrylate, 2-hydroxyethyl acrylate, 2-hydroxypropyl acrylate, butanediol monoacrylate, ethyldiglycol acrylate, lauryl acrylate, dimethylaminoethyl acrylate, and dihydrodicyclopentadienyl acrylate. The monomers can be functionalized by substituents. In some instances, the monomers include a functional group that can interact, e.g., through hydrogen bonding, with imprint moieties. Suitable functional groups include, for example, hydroxyl groups, amide groups, halogens, glycol groups, and amine groups. A suitable example of a bis-acrylate is 1,5-pentanediol bis(α-acetamido acrylate). A suitable example of an aryl methacrylate is benzyl methacrylate.

Nylons, for example, Nylon 6, Nylon 66, Nylon 12, and Nylon 612 can be used. Nylon 66 has a flexural modulus of about 450,000 psi, dry as molded (73° F., ASTM D790). Polyacrylates can be used, for example, polymethylacrylate, which has a flexural modulus of about 270,000 psi. Polyesters, for example, polyethylene terephthalate can be used. Polycarbonates can also be used, and have a flexural modulus of about 350,000 psi.

The monomers can be selected to have strong adhesion properties when copolymerized on, for example, a gold or other metal surface of a microbalance chip, e.g., a quartz crystal microbalance (QCM) chip. For example, the monomers can be of a general structure (CH3CONH)C(═CH2)COO(CH2)iOCOC(═CH2)(NHCOCH3), where i is, e.g., 4, 5, 8, 10, 20, or more, e.g. 100. The monomers can be polymerized, or copolymerized, under UV curing conditions, or by simple heating. In some instances, cross-linked polymers result.

The polymers have a flexural modulus of at least 150,000 psi. A polymer's stiffness when flexed (flexural modulus) is determined under 3 point loading conditions, as described in ASTM D790, the entire contents of which is hereby incorporated by reference. Specimens that are 3.2 mm×12.7 mm×64 mm (0.125 inch×0.5 inch×2.5 inch) are cast or molded, and conditioned as described in ASTM D790. An Instron® Universal Tester with flexural test fixtures is used for the testing procedure. The specimen is placed on a support span at room temperature (73° F.), and a load is applied to the center by a loading nose, producing three point bending data. The flexural modulus is calculated from the resulting stress/strain curve, as described in ASTM D790.

Under these ASTM D790 conditions, the flexural modulus of poly methyl methacrylate is about 270,000 psi. The flexural modulus of Nylon 66 is about 450,000 psi, dry as molded (73° F., ASTM D790). Some polycarbonates have a flexural modulus of about 350,000 psi.

Examples of imprint moieties that can be imprinted on the surface of the thin polymer film include bacterial cells, which have a diameter (overall size) of about 1 micron, and red blood cells, which have a diameter (overall size) of about 7 microns. Other imprint moieties include fungal, plant, mammalian cells, e.g., human cells, and virus particles.

The films are very thin and can be from about 100 nm to about 2 microns thick and typically have a thickness less than about one-third the diameter of an imprint moiety or other moiety to be imprinted. The film thickness can be about 10, 20, 25, 30, or 35 percent of the diameter of an imprint moiety. For example, the thickness of the film can be 100 to 1000 nm, 250 to 750, or 500 nm. The concentration of imprint moieties used to initially imprint the film can be high, e.g., 10⁷ to about 10⁹ moeities/mL, or can be equivalent to that found in environmental or bodily samples. The concentration can be 500 cells/mL, or higher, providing the ability to detect the existence of imprint moieties at environmentally relevant concentrations without further sample treatment, e.g., concentrating or purification of the sample. The minimum concentration of the imprint moieties that can be detected during imprinting (recapture of the imprint moieties) can be low, for example, in the range of 100 cells/mL to 1000 cells/mL.

In certain embodiments, polymer films are imprinted with a relatively large number of binding sites for specific imprint moieties, e.g., cells, so that low concentrations of an imprint moiety can be detected. The number of indentations or binding sites can be, e.g., 20,000 to 150,000 sites/cm² or more, e.g., 30,000, 40,000, 60,000, 70,000, 90,000, or more, e.g., 100,000 sites/cm². The number of indentations can be conviently determined using SEM.

In some embodiments, the QCM can detect the imprint moiety in a liquid sample at a minimum concentration of at least as low as 1000 imprint moieties per milliliter. This minimum concentration level can be measured by determining signal strength above background with a series of standard dilutions.

General Method of Making

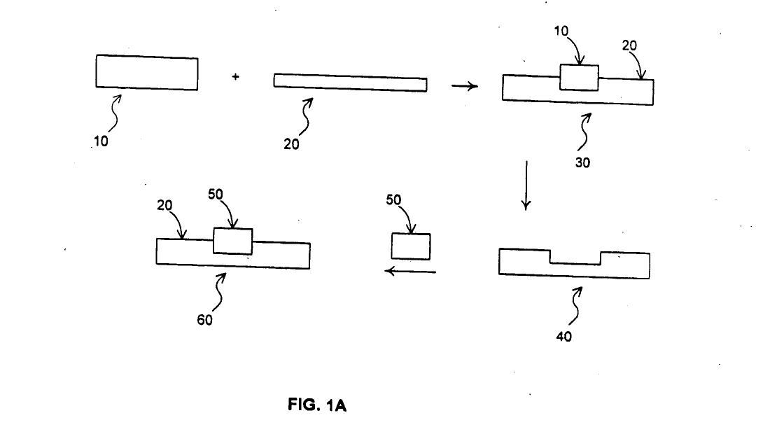

FIG. 1a, schematically illustrates how an imprint moiety 10 is contacted with a monomer layer 20 followed by polymerization to form the thin polymer film complex 30. The surface of imprint moiety 10 forms an imprint, e.g., a cavity, or indentation, within the liquid monomer layer 20 to form a complex 30. The monomers in the monomer layer 20 are then treated, or simply allowed to undergo a change in physical state (e.g., polymerization) to form a solid or semisolid polymer such that the changed form is capable of retaining shaped binding sites that can later specifically bind to imprint moiety 10. Removal of imprint moiety 10 from the solidified complex 30 yields an imprinted thin polymer film 40. Film 40 includes binding sites, which complement the topography of a portion of the surface of each imprint moiety 10.

To create the imprinted thin film, the monomer layer 20 is deposited onto a surface of a substrate. A layer of imprint moieties 10 can then be applied on the deposited monomer layer followed by polymerization, for example, thermal polymerization at 150 to 200° C. Other methods of polymerizing monomers can be, for example, free radical, UV, anionic, suspension, cationic, electro-polymerization, e-beam or gamma ray polymerization, and condensation polymerization. Next, after the polymer has set, the imprint moieties are removed from the imprint moiety-thin polymer film complex 30, e.g., by a lysis cocktail, e.g., a mixture of lysozyme, mutanolysin and lysostaphin available from SIGMA Chemicals, which causes cell disruption with a combination of detergents and hydrolytic enzymes. Residual cell debris is removed from the film by rinsing with a solvent, e.g., methylene chloride. The resulting imprinted thin polymer film 40 has specific structural binding sites imprinted on the surface of the film 40.

The monomers and imprint moieties can be deposited onto the surface by a variety of methods. For example, monomers, and optionally a crosslinking agent, for example, peroxides or azo compounds, and solvent, such as methylene chloride, tetrahydrofuran, or chloroform, can be mixed and applied to a surface to form a coated surface. The monomers can be applied to a surface, e.g., by spin coating, dip coating, spraying, or vaporization techniques. Next, an aqueous imprint moiety solution can be applied to the coated surface by spin coating, dip coating, spraying, or vaporization techniques. As noted above, polymerization of the monomers in the presence of the imprint moiety solution can occur by any polymerization method.

For example, any eukaryotic or prokaryotic cell can be used as an imprint moiety. Examples of imprint moieties, include, but are not limited to, mammalian cells, bacteria, spores (e.g., anthrax), fungi, yeast cells, molds, or viral particles. A naturally occurring normal, diseased, or genetically engineered imprint moiety can be used. In some embodiments, transformed cells or tumor cell lines can be used as imprint moieties. Suitable examples of bacterial cells to be used as imprint moieties include E. coli (Gram negative rods), Staphylococcus aureus (Gram-positive spheres), and Bacillus megaterium (Gram-positive rods). Mammalian cells can be, for example, endothelial cells or epithelial cells. Human cells can be used, e.g., from a biopsy.

Methods of Detecting Specific Imprint Moieties

The imprinted polymer films can be used to detect and/or characterize specific imprint moieties using a variety of detectors. Referring to FIG. 1a, film 40 can be contacted with a second imprint moiety 50 of the same type as imprint moiety 10 to capture the second imprint moiety to form an imprint moiety-selective polymer film complex 60. A detection device, or sensor, can be used to analyze complex 60. For example, the film 40 can be bound to the surface, e.g., formed or polymerized directly on the surface, of a conducting element of, for example, a sensor. The binding of the imprinted imprint moiety can be detected by a change in signal of the sensor. For example, when the sensor is a quartz crystal microbalance (“QCM”) chip, the QCM detector can detect increases in mass and shear at the surface resulting in a decreased frequency of the piezoelectric vibration of the QCM chip. One useful detector is a quartz crystal microbalance (QCM) for frequencies in the lower MHz range. Another detector is a surface acoustic wave resonator for frequencies up to 2.5 GHz. In each case, the imprinted polymer films are attached to the detector. When imprint moieties selectively bind to the imprinted thin polymer film, they produce a detectable change in the mass of the film. This change in mass is correlated to the change in frequency or resistance of the detector. Details of the QCM have been described, e.g., in Wegener et al., Biophysical Journal, 78: 2821-2833 (2000).

Characterization of Thin Polymer Films

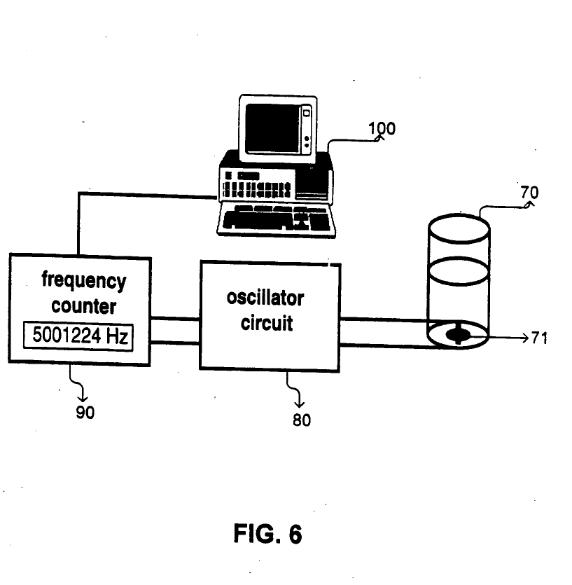

FIG. 6 shows a detection apparatus including a sensor, for example, a quartz crystal microbalance 70, an oscillator circuit 80, a frequency counter 90, and a computer 100. The QCM 70 includes a conducting element, for example, a QCM chip 71. The QCM is coupled to the oscillator circuit 80. The QCM 70 detects a change in mass balance of the thin polymer film surface coated on the chip 71 upon binding of an imprinted imprint moiety, and transmits the signal as a frequency response to the oscillator circuit 80. The oscillator circuit 80 is coupled to a frequency counter 90 that measures a frequency change. The frequency change from the frequency counter 90 is transmitted to, and analyzed by a computer 100, using standard techniques, and software.

Uses of Thin Polymer Films

Detection of Imprint Moieties

1. Environmental Contaminants Monitoring

The imprinted thin polymer film 40 can be placed on a biosensor for in-line, rapid and accurate quantification for natural, and bio-engineered imprint moieties. The imprint moieties can be present in naturally occurring samples, or can be artificially synthesized. Imprint moieties can be tested by (1) applying a monomer mixture to a sensor of a detector, for example, a conducting element of a QCM chip, (2) applying a first, target imprint moiety solution to the monomer mixture; (3) polymerizing the monomer mixture in the presence of the target imprint moiety solution, removing the imprint moieties to form an imprinted polymer surface; and monitoring the interaction of the polymer thin film surface with a test or sample solution by a change in the response as monitored by the detector. Based on the selectivity of the imprinted thin polymer film surface, target imprint moieties in the sample will bind to the surface of the film enabling detection of the target imprint moieties.

In one embodiment, the imprinted thin polymer film can be used in handheld detection devices as a first line-of-defense by military personnel or in the initial investigation of bio-terrorist attacks. The imprinted thin polymer film can be used by personnel, e.g., military, security, or medical personnel, to rapidly, and selectively detect potentially harmful concentrations of biowarfare agents, e.g., anthrax, smallpox, or botulinum. A series of sensors each uniquely specific to a different biowarfare agent can be exposed to a zone of contamination and read on-line in a hand-held device or in a nearby support vehicle. For example, swabs of surfaces in mail handling facilities or mailrooms can be mixed into an aqueous solution, and applied to thin polymer films imprinted with imprint moieties, or spores of anthrax bacteria. The ability to detect environmentally relevant concentrations (e.g., 100 to 500 imprint moieties/mL, or 500 to 1000 imprint moieties/mL) arises from the fact that the films are thin, thus improving sensitivity.

2. Industrial Process and Safety Control in Pharmaceuticals, and Food Processing

Control of pathogenic imprint moieties such as staphylococus, clostridinijum, E. coli, cryptosporidium, and other microbes is desired in industrial processing. The imprinted thin polymer films can be used in monitoring routine quality control for clinical testing, food processing, and water safety for determining the presence of any bacterial imprint moieties, especially of potential pathogenic imprint moieties. For example, an array of imprinted thin polymer films, each film being selective for a specific cell, can be placed on an indicator sheet. A swab of a surface from, for example, a food processing machine, can be applied to the array of imprinted thin polymer films to test for presence of pathogenic imprint moieties, for example, Staphylococcus aureus in clinical settings, Listeria monocytogenes, or Clostridium botulinum in food processing, E. coli, or cryptosporidium in routine water testing.

3. Clinical Diagnostics for Rapid Detection of Pathogens in Bodily Fluids (e.g., During Emergency Surgery)

There is increasing interest in sensors that can rapidly detect the presence of pathogens in bodily fluids. For example, an array of imprinted thin polymer films, each film being selective to a specific type of pathogen, can be placed on an indicator sheet. A bodily fluid can be tested for the presence of pathogens by placing the fluid on the array and testing the detector response to binding of a pathogen to the array. The rapid detection of pathogens in bodily fluids can allow a surgical team to estimate the potential for infection, and to evaluate the risk of steps that might support the spread of an infectious agent. Non-urgent detection such as the presence of certain bacterial strains in saliva can aid in diagnosis and in the choice of antimicrobial therapy (e.g., Streptococcus mutans in early childhood cavities). In cases of bioterrorism, one can test the presence of pathogens or their spores concentrated in nasal or oral saliva after a period of breathing contaminated air.

4. Routine Quality Control and Screening Tools for Hospital Contamination

Routine quality control for detection of contamination of surfaces, for example, hospital surfaces, can be carried out at environmentally relevant levels using imprinted thin polymer films in real time, allowing detection of a low concentration of imprint moieties such as bacteria. Sensors including imprinted thin polymer films can be robust and stable in extreme conditions such as acids, bases, solvents, and at high temperature and pressure. Hospital outbreaks of pathogenic bacterial strains that are resistant to antibiotics are a potential danger for immuno-compromised patients. This is especially dangerous when new resistance against formerly strong antibiotics is found (e.g., against Vancomycin). The ability to see results of screening tests of hospital areas immediately when the test is done can be invaluable for timely decision making.

5. Selective Sensors for Drug Testing

Sensors for rapid detection, and presence of drugs in samples can be manufactured using imprinted thin polymer films. Examples of sensors for rapid detection are described in various U.S. Patents.

6. Simultaneous Detection of Drug and Imprint Moiety

The imprinted thin polymer films can be used to test the effects of drugs on imprint moieties, such as cells. For example, imprint moieties attached to imprinted thin polymer films can act as biosensors. The interaction of a specific drug to the bound cells, e.g., cell lysis, can be studied to determine toxicity of the drug to the cells.

7. Recognition of Biomolecules

Proteins can be detected and quantified with high specificity in diagnostic assays and for biochemical research. Surface templation of proteins can aid in this approach, and reduce cost and duration of detection.

8. Kinetic Measurement of Cellular Growth and Metabolic Functions

Imprinted thin polymer films bound to, for example, a tumor cancerous cell, can be used to study the growth of these imprint moieties in a particular medium, providing insight into the metabolic characteristics of these imprint moieties. Cell growth and the inhibition of cell growth, for example, can be determined through changes in biomass weight of the imprinted thin polymer films.

9. Optical Detectors of Biofilms

Imprinted thin polymer films can be used to create transparent imprint moiety contact surfaces on optical detectors to warn of biofilm formation before detrimental consequences can occur in a particular system being monitored, e.g., a catheter. Biofilm formation can be locally accelerated by providing a preferred attachment surface overlaying an optical detector, thus providing an early-warning system since imprint moieties will grow first on the detector surface. Clean line systems (e.g., from catheters to industrial production lines) often run the risk of biofilm formation. Biofilm formation on the detector surface is seen as a dimming of incoming light. Thus, the biofilm formation can be detected, and detrimental consequences prevented.

Localizing of Imprint Moieties

1. Surface Supported Biocatalysts

Biologically friendly attachment sites can be created using imprinted thin polymer films to support recognition by imprint moieties growing as biofilms for utilization in production biotechnology. Many imprint moieties such as cell lines can have increased productivity when grown attached to a surface. Surface attachment can be provided, for example, for an imprint moiety of the biofilm forming imprint moieties. This can drastically increase, or enhance biofilm formation, and production of biocatalytic biofilms is shortened.

2. Binding Sites in Implantable Medical Devices

Imprinted thin polymer films can be used to attract, or localize mammalian cells to the surface of implantable medical devices, such as metallic or plastic body implants, for example, prostheses, such as hip joints, and related orthopedic devices, artificial blood vessels, pacemakers, and heart assist devices. These cells can be made to grow, or proliferate faster by attaching a thin polymer film surface with structured attachment sites to a surface an implant. For example, if the implant is selectively covered with a film that was previously imprinted with a culture of cells grown from the site of the future implant, the newly growing cells can find the mirror image of their surface structure leading to stimulation of the cells to grow faster, or to create a stronger connection to the implant.

3. Whole Cell Biosensors

The new thin polymer films, once imprinted with a particular cell, can be used to create whole cell biosensors by applying specific cells in a desired density onto the surface of the film before, or after it has been attached to a QCM sensor.

### EXAMPLES

The invention is further described in the following examples, which do not limit the scope of the invention described in the claims.

### Example 1

### Synthesis of a Monomer

A general synthetic procedure for α,ω-alkanediol bis(α-acetamido acrylate) is described. A mixture of α-acetamidoacrylic acid (0,016 mol, 2.064 g), α,ω-alkanedibromide (0.06 mol), K₂CO₃ (1.8 g) and DMSO (20 mL) was stirred at room temperature for four days. Deionized water (930 mL) was introduced and the resulting mixture was extracted with chloroform (30 mL×2). The chloroform phases were collected and washed with water (15 mL×3). The chloroform phase was dried over magnesium sulfate. Chloroform was evaporated by rotavapor and a solid was obtained. The remaining solvent was further removed under vacuum at room temperature to give a white solid product. For example, the monomer 1,4-butanediol bis(α-acetamido acrylate) was obtained in 85% yield. The ¹H NMR (CDCl₃, 300 MHz) showed δ(ppm)=1.76 (m, 4H, CO₂CH₂CH₂), 2.05 (s, 6H,CH₃), 4.20 (t, 4H, CO₂CH₂CH₂), 5.78 (s, 2H, vinyl CH), 6.52 (s, 2H, vinyl CH), 7.63 (s, 2H,NH). The monomer 1,5-pentanediol bis(α-acetamido acrylate) was prepared in 61% yield. The ¹H NMR (CDCl₃, 300 MHz) showed δ (ppm)=1.43 (p, 2H, CO₂CH₂CH₂CH₂), 1.72 (p, 4H,CO₂CH₂CH₂), 2.06 (s, 6H, CH₃), 4.20 (t, 4H, CO₂CH₂CH₂), 5.79 (s, 2H, vinyl CH), 6.53 (s, 2H, vinyl CH), 7.68 (s, 2H, NH). For example, the monomer 1,8-octanediol bis(α-acetamido acrylate) was obtained in 91% yield. The ¹H NMR (CDCl₃, 300 MHz) showed δ (ppm)=1.28 (m, 8H, CO₂CH₂CH₂CH₂), 1.64, (p, 4H, CO₂CH₂CH₂), 2.05 (s, 6H,CH₃), 4.18 (t, 4H, CO₂CH₂CH₂), 5.79 (s, 2H, vinyl CH), 6.52 (s, 2H, vinyl CH), 7.68 (s, 2H, NH).

### Example 2

### Biological Cell Preparation and Measurement

Pure culture cells were grown aerobically on an orbital shaker at 220 rpm to mid log phase in phosphate buffered rich medium (Luria-Bertani broth). A measured volume was harvested and washed once by mild centrifugation (4,000×g) in sterile saline solution (0.9% NaCl). Cell motility was inactivated for direct cell counting with Norm's-Powell solution (phosphate buffered SDS with 5% (vol/vol) of 37% Formaldehyde, pH 7.4). Cell concentration was determined in a Petroff-Hauser chamber using the average of 25 counts from 16 squares each. Imprinting and measuring were done with cell suspensions adjusted to approximately 500 cells/ml in sterile saline solution.

### Example 3

### Surface Imprinting of Thin Polymer Films

Referring to FIG. 1b, an imprinted thin polymer film was prepared by spin coating 20 μl of a CH₂Cl₂, (10 mL) solution containing 1,5-pentanediol bis(α-acetamido acrylate) (25.9 mg, 0.08 mmol), and benzyl acrylate (0.03 ml; 0.02 mmol, 80/20 mol ratio), onto a QCM chip (International Crystal Manufacturing, Oklahoma City, Okla.) for 30 seconds at 7500 rpm. A solution (100 μL) of bacterial cells was immediately spun coat onto the same QCM chip. The chip was then heated at 45° C. for 2 hours in air to polymerize the monomers. Cells were partially removed from the polymer film surface by a lysis cocktail (mixture of lysozyme, mutanolysin and lysostaphin available from SIGMA Chemicals, 10 mg/ml, 25 μg/ml and 0.5U/μl final concentration, respectively; 90 minutes at 37° C.) in a suitable volume of lysis buffer (10 mM Tris HCI (pH 8.5), 5 mM EDTA). The lysis cocktail and cell remnants were washed off with saline followed by methylene chloride, and the QCM chip was stored in sterile saline solution at 4° C. in the dark until use.

### Example 4

### Surface Characterization by Scanning Electron Microscopy

Scanning Electron Microscopy (“SEM”) was used to view surface features from the imprinting process. FIGS. 5a-d show SEM micrographs that illustrate a surface of the thin polymer film alone (5a), a surface that was polymerized in the presence of E. coli (“EC”) (5b), a surface after lysis (5c), and a surface after sensing occurred (after binding of the cells) (5d). FIG. 5c shows a divot or cavity (the binding site) left by the cells after lysis occurred. Binding of a E. coli cell is shown in FIG. 5d. The captured cell in this view was at an angle normal to the surface, which indicates that the entire cell need not bind to cause a signal change. SEM showed that the number of indentations was approximately 85,000 indentations/cm² .

### Example 5

### Quartz Crystal Microbalance (QCM) Analysis

QCM chips with thin polymer films were exposed to specific cell solutions at concentrations of approximately 500 cells/mL, and the QCM response in resonant frequency and admittance was measured as a function of time using a home built QCM recorder and a personal computer. FIG. 6 shows an experimental setup for detection using a QCM chip.

The electronic circuit was based on an active bridge oscillator circuit. An external frequency counter 90 was used, but can be replaced by a dedicated high-speed microprocessor circuit. The QCM 70 has an oscillator circuit 80 that is functional for a wide variety of analyte/QCM coating conditions. Typical oscillator circuits provide enough crystal drive current to oscillate the crystal in air, as the common function for crystals is for use as a time base. When a crystal was coated with a sensing material and was in contact with water, extra loads dampened the crystal and prevented it from oscillating. A special variable drive oscillator circuit was employed that sensed the crystal load and provided sufficient current to drive the loaded crystal without undo stress to the crystal.

For water-borne use, an O-ring was positioned around a gold electrode on top of the QCM chip. The sample stream was confined to the inside of the O-ring for two reasons: (1) metal ions in the sample stream can affix to metallic surfaces on the QCM, increasing mass and creating false positive readings; and (2) there was a benefit to minimizing the electronic damping effect of totally submerging the QCM in the sample stream. Finally, the housing surrounding the QCM was machined from a Delrin® block, with Delrin being used because of its inert properties and machinability. Alternatively, a molded silicon rubber housing can be used, eliminating the need for an O-ring.

### Example 6

### Selective Binding of Specific Cells

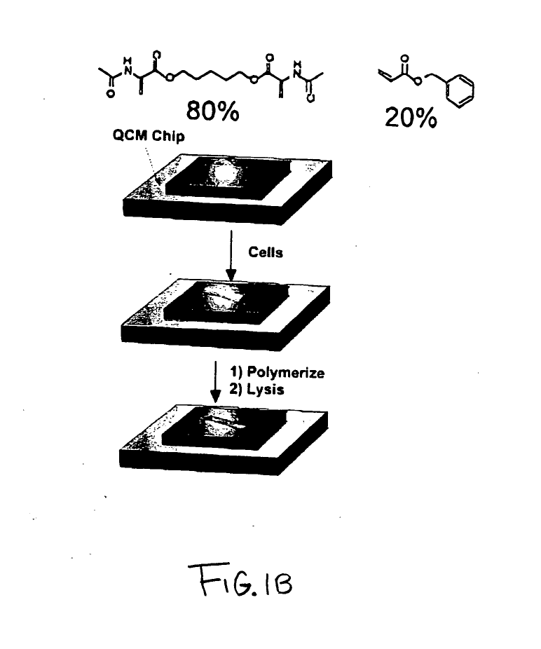

Three cell lines were examined to determine selectivity of the thin polymer films. Systematic variations of cell properties (cell wall structure, size, and overall geometry) were tested to provide a method to determine selectivity as a measure of the frequency response of the QCM to cell selectivity of imprinted and unimprinted cell types. Referring to FIGS. 2a-c, selectivity was evidenced by the large frequency shift from an equilibrium with saline upon addition of the selected cell (from zero to about −1500 Hz in FIG. 2a), as opposed to the small (of about −200 Hz in FIG. 2a) shift observed caused by unimprinted cells of varying size, shape, and cell wall composition. Cell lines of E. coli (Gram negative rods), Staphylococcus aureus (Gram-positive spheres), and Bacillus megaterium (Gram positive rods) were used. Each cell was imprinted on a separate, thin polymer film. The response of the conducting element of the sensor was measured for imprinted cells versus unimprinted cells. For example, in FIG. 2a, the imprinted cell was E. coli (EC). The unimprinted cells were Staphylococcus aureus (Gram-positive spheres), and Bacillus megaterium (Gram positive rods). Referring to FIG. 2a, the response of the sensor shows that selective cellular binding was observed for E. coli (EC-EC) over the unimprinted cells (EC-SA) and (EC-BM), indicating that the polymerization and lysis process generated a molecular imprint of E. coli (EC-EC) in the thin polymer film.

Referring to FIG. 2b, the imprinted cell was Bacillus megaterium. The unimprinted cells were Staphylococcus aureus, and E. coli. In FIG. 2b, the responses were measured for Bacillus megaterium, and the unimprinted Staphylococcus aureus (Gram-positive spheres) and E. coli. Referring to FIG. 2b, the decrease from zero to about −1000 Hz after about 20 minutes, and a steady drop to about −4200 after 250 minutes show that selective cellular binding was observed for the Bacillus megaterium (BM-BM) over the unimprinted cells (BM-SA) and (BM-EC), indicating that the polymerization and lysis process generated a molecular imprint of Bacillus megaterium in the thin polymer film.

Similarly, in FIG. 2c, the imprinted cell was Staphylococcus aureus. In FIG. 2c, the response was measured for Staphylococcus aureus (Gram-positive spheres), and unimprinted Bacillus megaterium, and E. coli. Referring to FIG. 2c, the frequency drop to about −1000 Hz after 20 minutes, and subsequent decline to about −3000 Hz after 200 minutes, show that selective cellular binding was observed for the Staphylococcus aureus (SA-SA) over the unimprinted cells (SA-EC) and (SA-BM), indicating that the polymerization and lysis process generated a molecular imprint of Staphylococcus aureus in the thin polymer film.

A QCM was used as described in Example 5 to measure the selectivity of the three different cell sensors. Thin polymer films imprinted for particular cells, for example, E. coli, a Gram-negative bacteria, do not appreciably bind to binding sites imprinted from Staphylococcus aureus or Bacillus megaterium. The observed binding (shown in FIG. 2a) of about −300 Hz for the unimprinted cell lines can be attributed to non-specific binding or a change in the viscosity of the saline solution. The results indicate that an appreciable signal can be generated in a few seconds, while ΔFₘₐₓ can be observed in about five to twenty minutes. A comparison of ΔFₘₐₓ (imprinted)/ΔFₘₐₓ (unimprinted) gave values, for example, of 24 for Bacillus megaterium, 10 for Staphylococcus aureus, and 4 for E. coli. The results show that selective cellular binding can be observed for the cell lines indicating that the polymerization and lyses process can produce molecular imprints in the thin polymer film.

### Example 7

### Analysis of Cell Properties in Solution

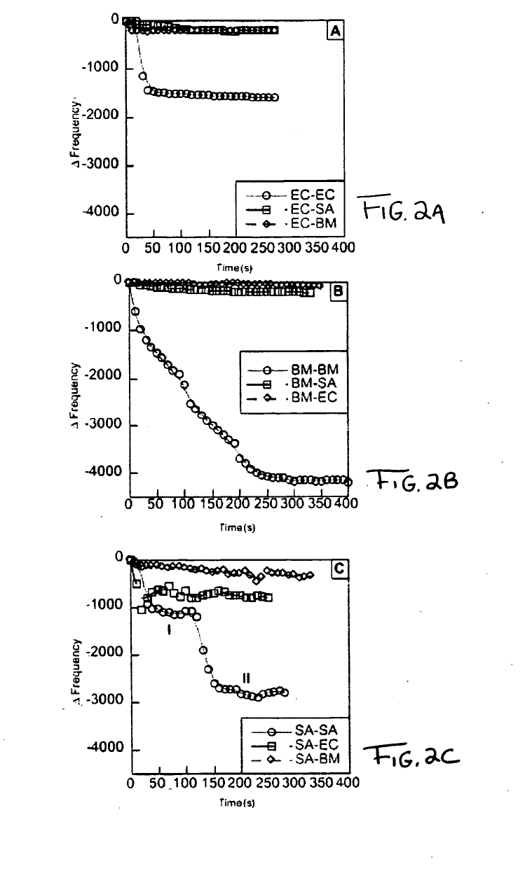

Referring to FIGS. 3a, 3b, and 3c, and FIGS. 2a, 2b, and 2c, the shape of response from the QCM was correlated to the imprint moiety's solution properties. For example, E. coli (“EA”) is known to exist as a single entity in solution (FIG. 3a). In comparison, B. megaterium (“BM”) cells are observed to form dimers and trimers in solution (FIG. 3b). This phenomenon explains the shallower slope of the curve to the imprinted cells in FIG. 2b, as there may be prebinding equilibria present, and the cells may have to break association with each other in solution before they can tightly bind to the surface (See FIGS. 2a and 2b). In the case of S. aureus (“SA”) (FIG. 3c), several possibilities exist as S. aureus cells strongly aggregate in solution. The two-step QCM curve (FIG. 2c) suggests two distinct binding processes, the first (I) due to small aggregates or single cell binding, and the latter (II) due to large aggregates of cells binding to the sensor.

In the case of S. aureus (FIG. 2c), the signal in response to the addition of non-target cells was the highest. This can be due to the fact that large aggregates of S. aureus were imprinted, generating large binding sites, and binding site heterogeneity on the surface of the polymer. The maximum frequency shift (ΔFₘₐₓ) was also correlated to cellular properties. The B. megaterium cells are also much larger than E. coli cells (FIG. 3a), and the ΔFₘₐₓ for B. megaterium was much larger than ΔFₘₐₓ E. coli (−4500 versus-1500 Hz). The results correlate the binding of a specific imprint moiety to its shape in solution.

### Example 8

### Correlation of Cell Wall Structure to Thin Polymer Film Selectivity

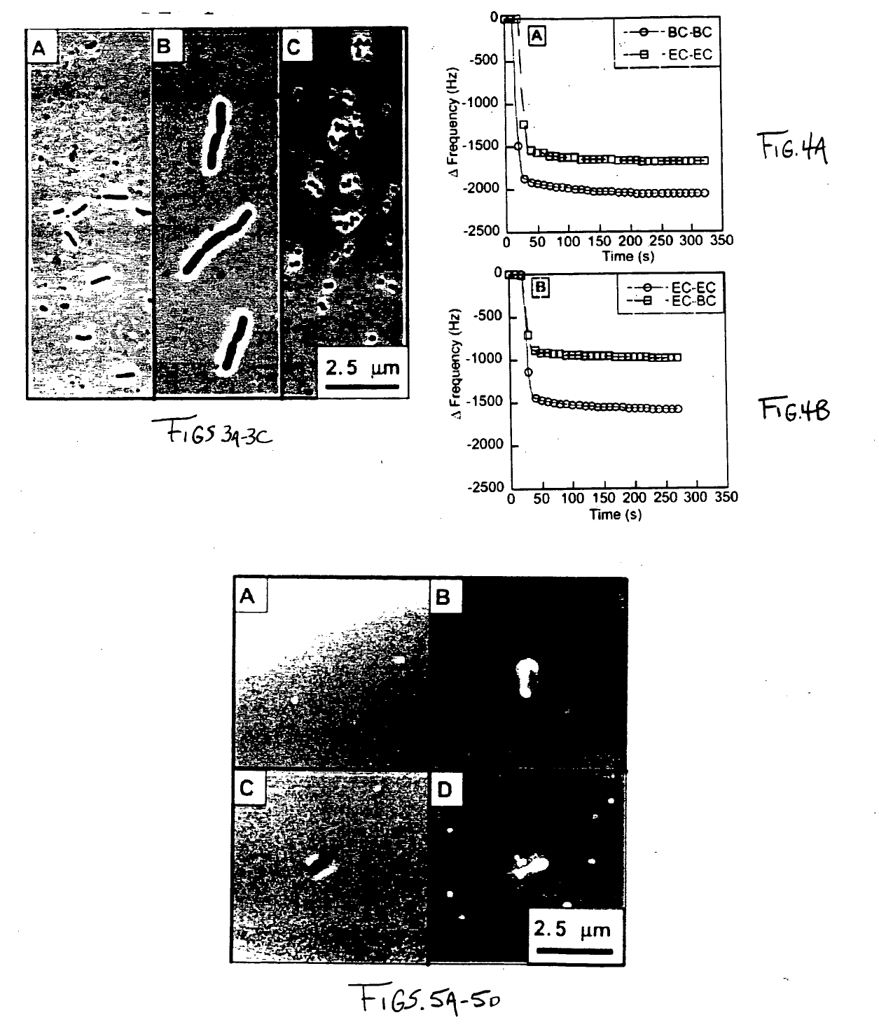

The nature of the imprinted thin polymer films' interaction with the incoming cells was examined by imprinting thin polymer films with two cells of similar shape, size, and solution characteristics, but different in cell wall structures. Bacillus cereus (BC) is a Gram-positive rod that exists as a single entity in solution and provides an excellent cell line for comparison to EC, a Gram-negative rod of similar size, which also exists as a single entity in solution. Removal of shape, size, and solution composition reduces the variable factor to differences in cell wall structure. Referring to FIGS. 4a, and 4b, the nature of the imprinted thin film polymer interaction with a cell of one cell wall structure was compared to the interaction of the thin film polymer with a cell of similar shape, and size, but different cell wall structure.

Referring to FIG. 4a, the QCM response curve of BC imprinted thin film polymer surface was measured for interaction with the imprinted cell BC (BC-BC) versus non-imprinted cell (BC-EC). Similarly, in FIG. 4b, the QCM response curve of EC imprinted thin film polymer surface is measured for interaction with the imprinted cell EC (EC-EC) versus non-imprinted cell (EC-BC). A modest amount of selectivity for imprint cell wall structure was observed between imprinted, and non-imprinted cells in FIGS. 4a, and 4b. A modest selectivity was observed in the ratio of the response of imprinted versus non-imprinted cells, ΔFₘₐₓ (imprinted)/ΔFₘₐₓ (non-imprinted). For example, in FIG. 4a, the ΔFₘₐₓ (imprinted)/ΔFₘₐₓ (unimprinted) was 1.3. In FIG. 4b, the ΔFₘₐₓ (imprinted)/ΔFₘₐₓ (non-imprinted) was 1.5. The results indicate that variation in cell wall, coupled with size, shape, and solution properties play a role in the recognition of imprint moieties by an imprinted thin polymer film.

### Other Embodiments

It is understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A thin film having a maximum thickness of 2 microns and comprising a polymer having a flexural modulus of at least 150,000 psi, wherein a surface of the film comprises a plurality of indentations, each indentation comprising a reverse impression of a portion of an outer surface of an imprint moiety, and wherein when the thin film is mounted on a quartz crystal microbalance (QCM), the QCM can detect the imprint moiety in a liquid sample at a minimum concentration of at least as low as 1000 imprint moieties per milliliter of sample.

2. The film of claim 1, wherein the imprint moiety is selected from the group consisting of bacteria, virus particles, animal cells, spores, plant cells, prokaryotic cells, and eukaryotic cells.

3. The film of claim 1, wherein the imprint moiety is selected from the group consisting of Escherichia coli, Staphylococcus aureus, and Bacillus megaterium.

4. The film of claim 1, wherein the maximum thickness is between about 100 nm and about 1500 nm.

5. The film of claim 1, wherein the flexural modulus is at least 200,000 psi.

6. The film of claim 1, wherein the flexural modulus is at least 250,000 psi.

7. The film of claim 1, wherein the QCM can detect the imprint moiety in a liquid sample at a minimum concentration of at least as low as 500 imprint moieties per milliliter of sample.

8. The film of claim 1, wherein the indentations have a maximum depth of from about 20% to about 40% of a largest dimension of the imprint moiety.

9. The film of claim 1, wherein the indentations on the surface number from about 40,000 to about 150,000 indentations/cm².

10. The film of claim 1, wherein the indentations on the surface number from about 60,000 to about 90,000 indentations/cm².

11. The film of claim 1, wherein the polymer comprises a bis-acrylate polymer.

12. The film of claim 1, wherein the polymer is selected from the group consisting of a methacrylate, an acrylate polymer, a nylon, a polyester, a polycarbonate, and mixtures thereof.

13. The film of claim 1, wherein the polymer is formed by polymerization of a mixture of 1,5-pentanediol bis(α-acetamido acrylate), and benzyl methacrylate or benzyl acrylate.

14. The film of claim 1, wherein the polymer is formed by polymerization of a monomer selected from the group consisting of acrylic acid, methyl acrylate, ethyl acrylate, butyl acrylate, 2-ethylhexyl acrylate, methacrylic acid, methylmethacrylate, isobutyl acrylate, tertiarybutyl acrylate, tertiarybutyl methacrylate, 2-hydroxyethyl acrylate, 2-hydroxypropyl acrylate, butanediol monoacrylate, ethyldiglycol acrylate, lauryl acrylate, dimethylaminoethyl acrylate, dihydrodicyclopentadienyl acrylate, adipic acid, hexamethylene diamine, carolactam, bisphenol, an organic diacid, a cyclic amide, ethylene glycol, terephthalic acid, an aromatic diacid, and mixtures thereof.

15. An implantable medical device comprising the thin film of claim 1 attached to at least a portion of its surface.

16. The medical device of claim 15, wherein the imprint moiety is a mammalian cell.

17. The medical device of claim 16, wherein the mammalian cell is from a mammal into which the medical device is to be implanted.

18. The medical device of claim 16, wherein the mammalian cell is an endothelial cell.

19. The medical device of claim 16, wherein the mammalian cell is a cartilage cell.

20. A biosensor comprising:
a microbalance comprising a conducting element; and
the thin film of claim 1, attached to a surface of the conducting element.

21. The biosensor of claim 20, wherein the microbalance is a quartz crystal microbalance.

22. The biosensor of claim 20, wherein the thickness of the film is from about 100 nm to 1500 nm.

23. The biosensor of claim 20, wherein the thickness of the film is less than 500 nm.

24. The biosensor of claim 20, wherein the imprint moiety is selected from the group consisting of a bacterium, virus particles, a cell from an animal, a spore, a plant cell, a prokaryotic cell, and a eukaryotic cell.

25. A method of detecting a target imprint moiety in a sample, the method comprising:
obtaining a biosensor of claim 20, wherein the thin film comprises indentations that are reverse impressions of the target imprint moiety to be detected;
applying a sample to the thin film on the biosensor under conditions that enable the thin film to selectively bind to any target imprint moieties in the sample, wherein the detector can detect the target imprint moiety at a minimum concentration of at least as low as 1000 imprint moieties per milliliter of sample; and
detecting a change in mass of the thin film, wherein an increase in mass of the thin film indicates the presence of the imprint moiety in the sample.

26. The method of claim 25, wherein the microbalance is a quartz crystal microbalance.

27. The method of claim 25, wherein the sample is blood, sputum, saliva, urine, or serum.

28. The method of claim 25, wherein the sample is water.

29. The method of claim 25, wherein the target imprint moiety is selected from the group consisting of bacteria, virus particles, animal cells, spores, plant cells, prokaryotic cells, and eukaryotic cells.

30. A method of making a thin film, the method comprising
polymerizing one or more monomers in the presence of a plurality of imprint moieties to form a polymer having a flex modulus of at least 150,000 psi;
forming a sheet of the polymer having a maximum thickness of 2 microns; and
removing the imprint moieties from a surface of the polymer sheet leaving a plurality of indentations on the surface, each indentation comprising a reverse impression of a portion of an outer surface of an imprint moiety;
wherein when the thin film is mounted on a quartz crystal microbalance (QCM), the QCM can detect the imprint moiety in a liquid sample at a minimum concentration of at least as low as 1000 imprint moieties per milliliter of sample.

31. The method of claim 30, wherein all of the imprint moieties are of the same type.

32. The method of claim 30, wherein the imprint moieties are of two or more different types.
